(19) United States
(12) Patent Application Publication
(10) Pub. No.: US 2024/0173142 A1
(43) Pub. Date: May 30, 2024

(54) IMPLANT ASSEMBLY

(71) Applicant: Biomet Manufacturing, LLC, Warsaw, IN (US)

(72) Inventor: Nathan A. Winslow, Scottsdale, AZ (US)

(21) Appl. No.: 18/507,971

(22) Filed: Nov. 13, 2023

Related U.S. Application Data
(60) Provisional application No. 63/428,273, filed on Nov. 28, 2022.

(51) Int. Cl. A61F 2/40; A61F 2/00; A61F 2/30; A61L 27/48

(52) U.S. Cl. CPC A61F 2/40; A61F 2/0095; A61F 2/30749; A61L 27/48; A61F 2002/30011; A61F 2002/30784; A61F 2002/3082; A61F 2002/30948; A61F 2002/30985; A61F 2310/00023

(57) ABSTRACT

An implant assembly can include a primary structure securable to a bone of a patient. The primary structure can include an adapted portion manufactured to match an anatomy of the bone of the patient. The adapted portion can also be engageable with the bone of the patient. The implant assembly can also include an attachment securable to the primary structure. The attachment can include an articular portion can define an implant articular surface. The implant articular surface can be engageable with a second bone of the patient. The attachment can also include a tailored portion extending from the articular portion. The tailored portion can be manufactured to match the anatomy of the bone of the patient and be engageable with the bone of the patient.

### CLAIM OF PRIORITY

This application claims the benefit of U.S. Provisional Patent Application Ser. No. 63/428,273, filed on Nov. 28, 2022, the benefit of priority of which is claimed hereby, and which is incorporated by reference herein in its entirety.

### BACKGROUND

The head of the humerus and the shallow socket of the scapula form a mobile unit called the glenohumeral (or shoulder) joint. The shoulder joint is held in place by various soft tissues, including muscles, tendons, and ligaments. Injuries, or wear, of the anatomy around the shoulder joint, can result in joint instability. Shoulder instability can make it difficult to raise your arm or complete everyday tasks. Sometimes, a surgical procedure is necessary to improve shoulder instability.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates a top view of an example of an implant assembly attached to a bone of a patient.

FIG. 2 illustrates an enlarged top view of an example of an implant assembly.

FIG. 3 illustrates a lateral view of an example of an implant assembly attached to a bone of a patient.

FIG. 4 illustrates a side view of an example of an implant assembly.

FIG. 5 is a block diagram illustrating a method of making an implant assembly.

FIG. 6 is a block diagram illustrating a method of installing an implant assembly into a patient.

### DETAILED DESCRIPTION

The glenoid when articulating with the humerus receives mechanical load that typically comes from the humeral head of the shoulder. This loading can lead to wear on the glenoid, or bone loss over time, often on the posterior aspect of the glenoid. The bone loss can result in instability of the shoulder joint. Shoulder joint instability can be mitigated using a coracoid autograft procedure. Specifically, the coracoid autograft procedure may help address bone loss by providing structural reinforcement to the posterior portion of the bone. An injury that causes damage to the boney covering of the glenoid can also necessitate a coracoid autograft procedure.

The inventors of the present disclosure have identified at least the following problems with the current method of resolving shoulder instability: 1) the autograft bone may not fit against the side of a glenoid well, which requires the autograft bone to be modified during insertion; 2) the graft may not be positioned optimally to resist the forces that are placed on the graft because of poor alignment with the bone of the patient; 3) loading issues may be present because the graft may not align correctly with the articular surface of the glenoid; 4) there can be difficulty securing the coracoid autograft because a surgeon can have poor visibility during screw placement; and 5) early failure of fixation method, resorption of the graft, or failure of the graft can occur because of poor fixation or unusual loading of the installed graft.

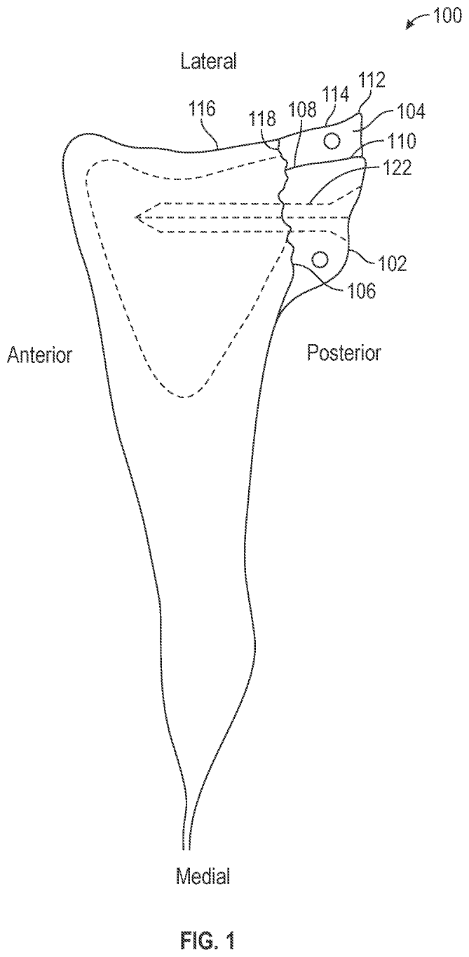

This disclosure discusses devices and methods that can help reduce at least the above-identified issues with the current methods to repair shoulder instability issues. The disclosed implant assembly can be designed to fit within a shoulder joint of a patient. The implant assembly can be customized for the patient based on a CT scan of a patient's bone. The shoulder assembly can have a primary structure made of a metallic material, and an attachment made of a non-metallic material. Both the primary structure and the attachment can be customized to fit against the natural anatomy of the bone such that there is no bone prep needed during the insertion of the device, and so that there is limited space or play between the implant assembly and the bone of the patient after the implant assembly is secured to the bone of the patient. As the implant assembly is fully customizable, fastener guides can be customized to alter the placement of the fasteners within the bone of the patient. Additionally, features can be added to the primary structure to aid in the attachment of soft tissue after the implant assembly is installed on the patient. The implant assembly can be designed such that the non-metallic portion is congruent with the patient's natural articular surface of the bone. As such, the implant assembly can improve longevity and function and can be more efficiently implanted into the patient than the current solutions. The implant assembly will be discussed in more detail below with reference to FIGS. 1-6.

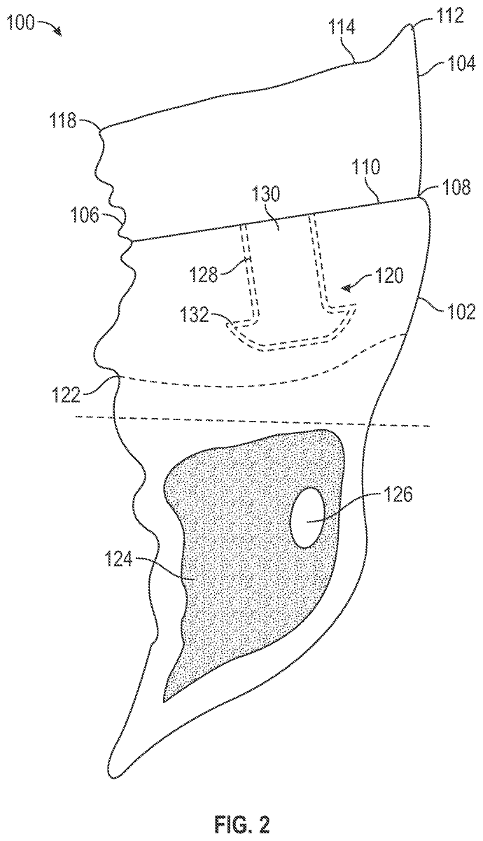

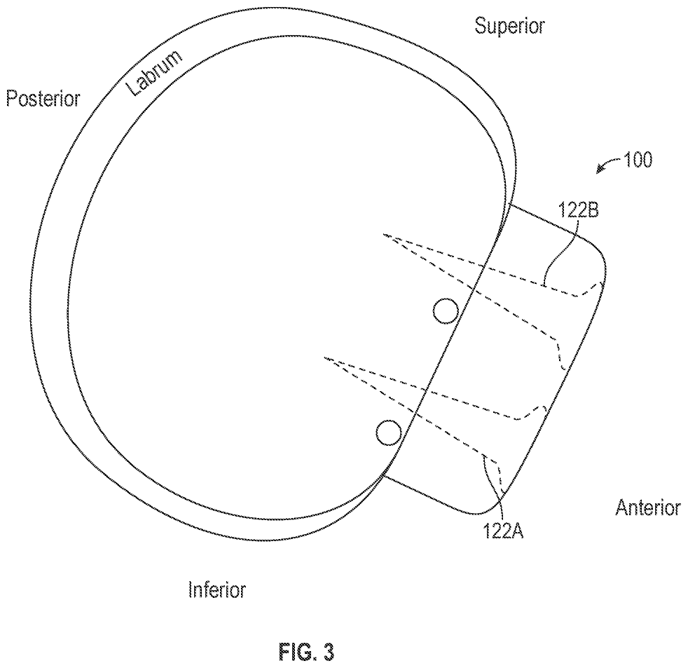

FIGS. 1-3 will be discussed together below. FIG. 1 illustrates an example of an implant assembly 100 attached to a bone of a patient. FIG. 2 illustrates a top perspective view of an example of the implant assembly 100. FIG. 3 illustrates a perspective lateral view of an example of the implant assembly 100 attached to a bone of a patient. FIG. 1 shows orientation indicators Medial, Lateral, Anterior, and Posterior. FIG. 3 shows orientation indicators Superior, Inferior, Posterior, and Anterior. The implant assembly 100 can include a primary structure 102, and an attachment 104.

The primary structure 102 can be securable to a bone (e.g., a scapula) of a patient. The primary structure 102 can be the portion of the implant assembly 100 that is securable to a bone of the patient. In an example, the primary structure 102 can be made of OsseoTi®, a porous metallic material that mimics the porous structure of human bones and facilitates tissue ingrowth and remodeling, vascularization, and very close attachment between bone and the metal. In another example, the primary structure 102 can be made of any material that is implantable into a patient and is biocompatible, such as one or more of titanium, UHMWPE, stainless steel, ceramic, cobalt-chromium, Polyether ether ketone (PEEK), or the like. The primary structure 102 can include an adapted portion 106 and a mating portion 108.

The attachment 104 can be securable to the primary structure 102. The attachment 104 can be made of vitamin E polyethylene. For example, the attachment 104 can be made of Vivacit-E®, a highly crosslinked polyethylene with antioxidant protection to meet the long-term performance needs of high-demand patients. In another example, the attachment 104 can be made of any material that is implantable into a patient and is biocompatible, such as one or more of ultra-high-molecular-weight polyethylene, Polytetrafluoroethylene (PTFE), ceramic, or the like.

The adapted portion 106 can be manufactured (e.g., sized and shaped) to match an anatomy of the bone of the patient and engageable with the bone of the patient. As shown in FIGS. 1 and 2, the adapted portion 106 can be formed or machined to align with the bone of the patient. Aligning the adapted portion 106 with the bone of the patient can provide support to the implant assembly 100 and can minimize play in the implant assembly 100 after the implant assembly 100 is installed on the bone of the patient. The support and minimized play provided by the adapted portion 106 can increase the durability and the performance of the implant assembly 100. For example, eliminating the play between the implant assembly 100 and the bone helps maintain an articular surface congruent to and matches the bone. Maintaining the articular surface helps eliminate stress concentrations and helps the bone function more naturally.

The mating portion 108 can extend away from the adapted portion 106. The mating portion 108 can be configured to receive attachments (e.g., the attachment 104). The mating portion 108 can provide more flexibility in the implant assembly 100 because a doctor or surgeon can select or build a customized attachment. Moreover, if something changes in the shoulder joint of the patient following completion of a primary procedure, the surgeon can order a new attachment (e.g., the attachment 104), remove the installed attachment, and replace the removed attachment with the ordered attachment, thus simplifying the revision procedure process. Moreover, the mating portion 108 can be configured to support attachments of the implant assembly 100. As discussed above, the mating portion 108 can be made from various metals, metallic alloys, or composites. The mating portion 108 can support a non-metallic attachment to help limit the warping or bending of the component.

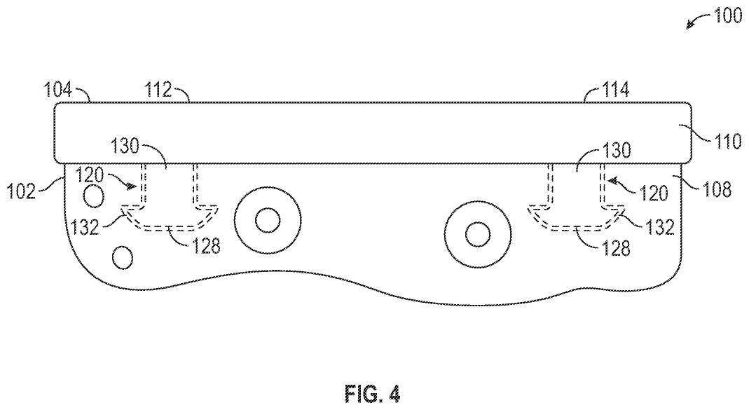

The attachment 104 can include a coupling portion 110, an articular portion 112, and a fitted portion 118. The coupling portion 110 can can be a planar surface engageable with the mating portion 108 of the primary structure 102. As such, the coupling portion 110 can be configured to attach to the mating portion 108 of the primary structure 102. The coupling portion 110 can be configured to transfer forces applied to the articular portion 112 to the mating portion 108 such that the mating portion 108 supports the articular portion 112. The coupling portion 110 will be discussed in more detail below with reference to FIG. 4.

The articular portion 112 can define an implant articular surface 114. The implant articular surface 114 can be engageable with a second bone (e.g., a head of a humerus) of the patient. In an example, the implant articular surface 114 can be congruent to and can match a bone articular surface of the bone of the patient to together form an improved articular surface 116 engageable with the second bone of the patient.

Changes to the articular portion 112 can change how the implant assembly 100 interacts with the second bone of the patient. For example, increasing a height of the articular portion 112 along the periphery of the articular portion 112 can increase support within the shoulder joint of the patient. The increased support within the shoulder joint of the patient can help reduce instability of the shoulder joint. In another example, decreasing the height of the articular portion 112 along the periphery of the articular portion 112 can provide flexibility and mobility of the shoulder of the patient. Therefore, the articular portion 112 can be customized to improve shoulder function of the patient.

The fitted portion 118 can extend between the coupling portion 110 and the articular portion 112. The fitted portion 118 can be manufactured to match the anatomy of the bone of the patient and can be engageable with the bone of the patient. As shown in FIGS. 1 and 2, the fitted portion 118 can be configured (e.g., sized or shaped) to align with the bone of the patient. Aligning the fitted portion 118 with the bone of the patient can provide support to the implant assembly 100 and can minimize play, such as relative movement, in the implant assembly 100 after the implant assembly 100 is installed on the bone of the patient. The support and minimized play provided by the fitted portion 118 can increase the durability and the performance of the implant assembly 100. For example, reducing the play between the implant assembly 100 and the bone helps maintain an articular surface congruent to and matches the bone. Maintaining the articular surface helps eliminate stress concentrations and helps the bone function more freely.

The primary structure 102 can further include an attachment channel 120 engageable with the attachment 104 to help secure the attachment 104 to the primary structure 102. The attachment channel 120 will be discussed in more detail below with reference to FIG. 4.

The primary structure 102 can also include an guide bore 122. The guide bore 122 can receive a fastener to attach the implant assembly 100 to the bone of the patient. As shown in FIG. 1 and FIG. 3, the guide bore 122 can extend through the primary structure 102 such as to direct the fastener into a bone vault of the bone of the patient (or can be planned to direct the fastener into the best quality bone).

As shown in FIG. 3, a first of the guide bore 122A can direct a first fastener in a direction that is substantially parallel to a second fastener directed by a second of the guide bore 122B. In another example, the guide bore 122 can direct the first and second fasteners in different directions to help seat and secure the primary structure 102 on the bone of the patient. For example, a first of the guide bore 122A can slightly guide a fastener in a superior direction while a second of the guide bore 122B can guide a fastener in a medial direction.

As shown in FIG. 2, the primary structure 102 can include a porous region 124. The porous region 124 can be included, such as attaching the soft tissue of the patient to the implant assembly 100. The porous region 124 can be located around the primary structure 102 to support soft tissue to attach and grow into the primary structure 102. The location of the porous region 124 can be customized based on the shoulder of the patient. For example, the porous region 124 can be located to aid in attaching different soft tissue to the primary structure 102. In another example, the surgeon can pre-operatively decide which soft tissue they would like to attach to the device and where the most appropriate soft tissue attachment location is on the device to accomplish the best results for the patient.

As shown in FIG. 2, the primary structure 102 can also include a suture canal 126. The suture canal 126 can be a bore or other hole extending at least partially through the primary structure 102. The suture canal 126 can be included such as to receive sutures at least partially therethrough to attach the soft tissue of the patient to the implant assembly 100. The suture canal 126 can be located around the primary structure 102 to provide support for soft tissue to attach and grow into the primary structure 102. The location of the suture canal 126 can be customized based on the shoulder of the patient. For example, the suture canal 126 can be located to aid in attaching different soft tissue to the primary structure 102. In examples, a surgeon can insert a juggerknot suture anchor, any other anchor, or the like, into the suture canal 126.

As also shown in FIG. 2, the primary structure 102 can include porous region 124, which can, together with the suture canal 126 to provide support and anchor points for soft tissue. In another example, the primary structure 102 can include the porous region 124 or the suture canal 126 based on the quality and shape of the soft tissue available to the surgeon.

FIG. 4 illustrates a side view of an example of an implant assembly 100. The attachment 104 can include a post 128 (also shown in phantom in FIG. 2). As shown in FIG. 4, the attachment channel 120 of the primary structure 102 can receive the post 128 of the attachment 104 to secure the attachment 104 to the primary structure 102 such that the mating portion 108 and the coupling portion 110 can be conterminous or are adjacent one another while the primary structure 102 and the attachment 104 are coupled to each other. When the post 128 is in the attachment channel 120, and the mating portion 108 and the coupling portion 110 are conterminous, the coupling portion 110 can transfer forces applied to the attachment 104 to the mating portion 108, such that the mating portion 108 and the primary structure 102 provide support for the coupling portion 110 and the attachment 104.

The post 128 can be inserted into, or otherwise engage with, the primary structure 102 to attach the primary structure 102 and the attachment 104. In one example, the post 128 can include a base 130 and a distal section 132. The base 130 can extend from the coupling portion 110 of the attachment 104. The distal section 132 can extend from the base 130. The distal section 132 can include a cross-sectional area greater than a cross-sectional area of the base 130 such that the distal section 132 limits separation of the post 128 within the attachment channel 120 after the post 128 is inserted into the attachment channel 120.

The attachment 104 can be coupled to the primary structure 102 using other methods. For example, the attachment 104 can include a dovetail complementary to a groove in the primary structure 102, such as to couple the attachment 104 and the primary structure 102. In such an example, a set screw, or other fasteners can also be used to help couple the attachment 104 and the primary structure 102. In another example, the attachment 104 can include a keel, the keel extending into a slot in the primary structure 102. The keel can extend through the primary structure 102 such that when the fastener is inserted into the guide bore 122, the fastener holds the attachment 104 and the primary structure 102 connected. In yet another example, the attachment 104 can be attached to the primary structure 102 in any way that can couple the primary structure 102 and the attachment 104.

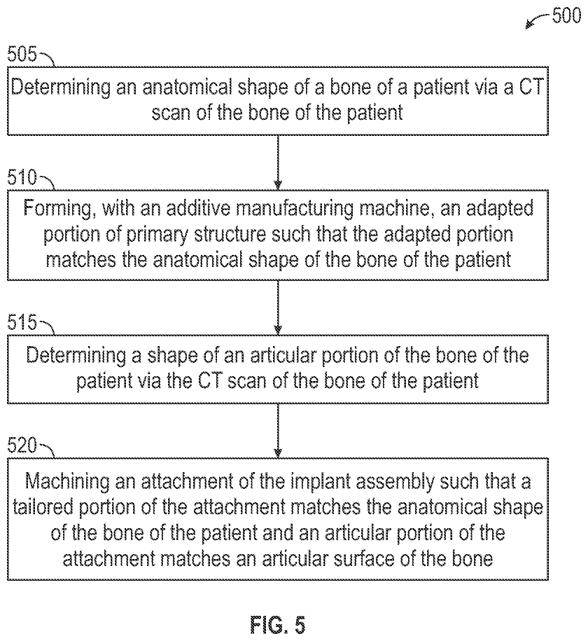

FIG. 5 illustrates a schematic view of the method 500, in accordance with at least one example of this disclosure. The method 500 can be a method of making an implant assembly (e.g., the implant assembly 100). More specific examples of the method 500 are discussed below. The steps or operations of the method 500 are illustrated in a particular order for convenience and clarity; many of the discussed operations can be performed in a different sequence or in parallel without materially impacting other operations. The method 500, as discussed, can include operations performed by multiple actors, devices, or systems. It is understood that subsets of the operations discussed in the method 500 can be attributable to a single actor, device, or system that could be considered a separate standalone process or method.

At operation 505, the method 500 can include determining an anatomical shape of a bone of a patient via a CT scan of the bone of the patient. In examples, a 3D CAD model, or any other model that can simulate the bone of the patient, can be used to design the implant assembly. For example, the model can be used to form an adapted portion (e.g., the adapted portion 106) of a primary structure (e.g., the primary structure 102), a customized, tailored, or fitted portion (e.g., the fitted portion 118) of an attachment (e.g., the attachment 104), and the articular portion (e.g., the implant articular surface 114 of the attachment). In examples, a 3D model of the implant assembly can also be made to compare the fit against the model of the bone of the patient. Moreover, the model of the implant assembly can be altered to determine a combination and configuration of soft tissue anchors (e.g., the porous region 124 and the suture canal 126, or any other soft tissue anchor). In such examples, finite element analysis can be used to compute the performance of the implant assembly with the modeled configuration.

At operation 510, the method 500 can include forming, with an additive manufacturing machine, an adapted portion (e.g., the adapted portion 106) of a primary structure (e.g., the primary structure 102) such that the adapted portion matches the anatomical shape of the bone of the patient. Here, the primary structure can be made using selective laser sintering, direct metal laser sintering, powder bed fusion, electron beam melting, laser material deposition, electron beam, or any other form of additive manufacturing or a combination thereof. After forming the primary structure with additive manufacturing, there may be additional manufacturing steps such as cleaning up any manufacturing debris or modifying any of the formed surfaces. For example, a wire brush or wheel can be used to clean the formed surfaces, or a Dremel or other abrasive bit can be used to modify the texture of the surface.

At operation 515, the method 500 can include determining a shape of an articular portion of the bone of the patient via the CT scan of the bone of the patient. Here, the shape of the articular portion can be used to design an articular portion (e.g., the articular portion 112) of an attachment (e.g., the attachment 104) of the implant assembly (e.g., the implant assembly 100) such as to verify that an articular surface of the implant is congruent to and matches the articular portion of the bone. The implant articular surface and the bone articular surface together form an improved articular surface (e.g., the improved articular surface 116) engageable with a second bone (e.g., the humerus) of the patient. The 3D model of the bone and the 3D model of the implant assembly can be used to verify that the attachment fits upon the primary structure, such as to align the articular surface of the bone and the articular surface of the implant (e.g., the implant articular surface 114).

At operation 520, the method 500 can include machining an attachment of the implant assembly such that a tailored portion of the attachment matches the anatomical shape of the bone of the patient and an articular portion of the attachment matches an articular surface of the bone. The attachment portion can be made from any non-metallic material insertable into a patient. The attachment portion can be machined to match the bone of the patient. In examples, a basic mold can be made for a starting shape of the attachment, and such a mold can be machined down to the shape and size required for the anatomical shape and size of the bone of the patient. The method 500 can also include packaging the primary structure and the attachment of the implant assembly within the packaging and sterilizing the implant assembly and the packaging.

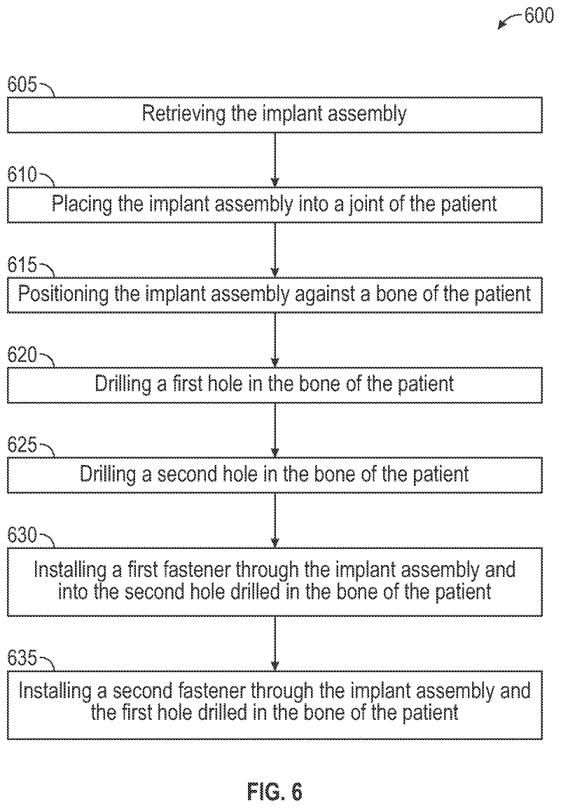

FIG. 6 illustrates a schematic view of the method 600, in accordance with at least one example of this disclosure. The method 600 can be a method of inserting an implant assembly into a patient. More specific examples of the method 600 are discussed below. The steps or operations of the method 600 are illustrated in a particular order for convenience and clarity; many of the discussed operations can be performed in a different sequence or in parallel without materially impacting other operations. The method 600, as discussed can include operations performed by multiple different actors, devices, or systems. It is understood that subsets of the operations discussed in the method 600 can be attributable to a single actor, device, or system could be considered a separate standalone process or method.

At operation 605, the method 600 can include retrieving the implant assembly. In examples, the implant assembly (e.g., the implant assembly 100) can be received from a manufacturer, and the implant assembly and the package that the implant assembly is packaged within can be sterilized and ready for insertion into the patient. In examples, the method 600 can include opening a package containing the implant assembly.

At operation 610, the method 600 can include placing the implant assembly into a joint of the patient. The implant assembly can be placed in a shoulder joint of the patient arthroscopically or through open surgery. In one example, the entire implant assembly can be placed into the joint of the patient. Here, the method 600 can also include coupling the primary structure to the attachment of the implant assembly by inserting an post of the attachment into an attachment channel of the primary structure before inserting the implant assembly into the joint of the patient. In another example, the primary structure can be placed into the patient.

At operation 615, the method 600 can include positioning the implant assembly against a bone of the patient. As such, the implant assembly can be placed against the bone such that the adapted portion of the primary structure aligns with the bone of the patient that the adapted portion was designed (via the CT scan and 3D model of the bone) to fit against.

At operation 620, the method 600 can include drilling a first hole in the bone of the patient. The primary structure of the implant assembly can be used as a guide for drilling the first hole. For example, an guide bore (e.g., the guide bore 122) can be used as a guide for the placement of the first hole in the bone of the patient. In another example, another guide can be used against the bone to ensure the placement of the holes in the bone will align with the guide bore of the primary structure. The method 600 can also include pinning the implant assembly within the first hole to hold the implant assembly steady while drilling a second hole within the bone of the patient.

At operation 625, the method 600 can include drilling the second hole in the bone of the patient. The primary structure of the implant assembly can be used as a guide for drilling the second hole. For example, an guide bore (e.g., the guide bore 122) can be used as a guide for the placement of the second hole in the bone of the patient. In another example, another guide can be used against the bone to ensure the placement of the holes in the bone will align with the guide bore of the primary structure. At operation 630, the method 600 can include installing a first fastener through the implant assembly and into the second hole drilled in the bone of the patient.

At operation 635, the method 600 can include installing a second fastener through the implant assembly and the first hole drilled in the bone of the patient. In one example, the method 600 can include attaching soft tissue to the implant assembly by inserting soft tissue through suture canal 126 in the implant assembly. In another example. the method 600 can include attaching soft tissue to the implant assembly by inserting soft tissue through a porous region 124 of the implant assembly.

### Additional Notes & Examples

Example 1 is an implant assembly comprising: a primary structure securable to a bone of a patient, the primary structure including: an adapted portion manufactured to match an anatomy of the bone of the patient and engageable with the bone of the patient; and a mating portion extending away from the adapted portion; and an attachment securable to the primary structure, the attachment including: a coupling portion engageable with the mating portion of the primary structure; an articular portion opposite the coupling portion, the articular portion defining an implant articular surface, the implant articular surface engageable with a second bone of the patient; and a tailored portion extending between the coupling portion and the articular portion, the tailored portion manufactured to match the anatomy of the bone of the patient and engageable with the bone of the patient.

In Example 2, the subject matter of Example 1 includes, wherein the implant articular surface is congruent to and matches a bone articular surface of the bone of the patient to together form an improved articular surface engageable with the second bone of the patient.

In Example 3, the subject matter of Examples 1-2 includes, wherein the attachment is made of a vitamin E polyethylene.

In Example 4, the subject matter of Examples 1-3 includes, wherein the attachment includes an post comprising: a base extending from the coupling portion; and a distal section extending from the base, wherein the distal section includes a cross-sectional area greater than a cross-sectional area of the base.

In Example 5, the subject matter of Example 4 includes, wherein the primary structure includes an attachment channel engageable with the post of the attachment to secure the attachment to the primary structure.

In Example 6, the subject matter of Example 5 includes, wherein the primary structure further comprises an guide bore, the guide bore configured to receive a fastener to attach the implant assembly to the bone of the patient.

In Example 7, the subject matter of Example 6 includes, wherein the guide bore extends through the primary structure such as to direct the fastener into a bone vault of the bone of the patient.

In Example 8, the subject matter of Examples 1-7 includes, wherein the primary structure comprises a porous region, the porous region configured to attach soft tissue of the patient to the implant assembly.

In Example 9, the subject matter of Examples 1-8 includes, wherein the primary structure comprises suture canal, the suture canal configured to receive sutures to attach soft tissue of the patient to the implant assembly.

In Example 10, the subject matter of Examples 1-9 includes, wherein the bone is a scapula and wherein the second bone is a humerus.

In Example 11, the subject matter of Examples 1-10 includes, wherein the primary structure is made of OsseoTi®.

Example 12 is an implant assembly comprising: a primary structure securable to a bone of a patient, the primary structure including: an adapted portion manufactured to match an anatomy of the bone of the patient and engageable with the bone of the patient; and an attachment securable to the primary structure, the attachment including: an articular portion defining an implant articular surface, the implant articular surface engageable with a second bone of the patient; and a tailored portion extending from the articular portion, the tailored portion manufactured to match the anatomy of the bone of the patient and engageable with the bone of the patient.

In Example 13, the subject matter of Example 12 includes, wherein the implant articular surface is congruent to and matches a bone articular surface of the bone of the patient to together form an improved articular surface engageable with the second bone of the patient.

In Example 14, the subject matter of Examples 12-13 includes, wherein the attachment includes an post comprising: a base extending from a periphery of the attachment; and a distal section extending from the base, wherein the distal section includes a cross-sectional area greater than a cross-sectional area of the base.

In Example 15, the subject matter of Example 14 includes, wherein the primary structure includes an attachment channel engageable with the post of the attachment to secure the attachment to the primary structure.

In Example 16, the subject matter of Example 15 includes, wherein the primary structure further comprises an guide bore, the guide bore configured to receive a fastener to attach the implant assembly to the bone of the patient.

In Example 17, the subject matter of Example 16 includes, wherein the guide bore extends through the primary structure such as to direct the fastener into a bone vault of the bone of the patient.

In Example 18, the subject matter of Examples 12-17 includes, wherein the primary structure comprises a porous region, the porous region configured to attach soft tissue of the patient to the implant assembly.

In Example 19, the subject matter of Examples 12-18 includes, wherein the primary structure comprises suture canal, the suture canal configured to receive sutures to attach soft tissue of the patient to the implant assembly.

Example 20 is a method of making an implant assembly comprising: determining an anatomical shape of a bone of a patient via a CT scan of the bone of the patient; forming, with an additive manufacturing machine, an adapted portion of a primary structure such that the adapted portion matches the anatomical shape of the bone of the patient; determining a shape of an articular portion of the bone of the patient via the CT scan of the bone of the patient; and machining an attachment of the implant assembly such that a tailored portion of the attachment matches the anatomical shape of the bone of the patient and an articular portion of the attachment matches an articular surface of the bone.

In Example 21, the subject matter of Example 20 includes, packaging the primary structure and the attachment of the implant assembly within packaging; and sterilization the implant assembly and the packaging.

Example 22 is a method of inserting an implant assembly into a patient comprising: retrieving the implant assembly; placing the implant assembly into a joint of the patient; positioning the implant assembly against a bone of the patient; drilling a first hole in the bone of the patient; drilling a second hole in the bone of the patient; installing a first fastener through the implant assembly and into the second hole drilled in the bone of the patient; and installing a second fastener through the implant assembly and the first hole drilled in the bone of the patient.

In Example 23, the subject matter of Example 22 includes, opening a package containing the implant assembly; and coupling an primary structure to an attachment of the implant assembly by inserting an post of the attachment into an attachment channel of the primary structure.

In Example 24, the subject matter of Examples 22-23 includes, pinning the implant assembly within the first hole to hold the implant assembly steady while drilling the second hole within the bone of the patient.

In Example 25, the subject matter of Examples 22-24 includes, attaching soft tissue to the implant assembly by inserting soft tissue through suture canal in the implant assembly.

In Example 26, the subject matter of Examples 22-25 includes, attaching soft tissue to the implant assembly by inserting soft tissue through a porous region of the implant assembly.

Example 27 is an apparatus comprising means to implement of any of Examples 1-26.

Example 28 is a system to implement of any of Examples 1-26.

Example 29 is a method to implement of any of Examples 1-26.

In Example 30, the apparatus, system, or method of any one or any combination of Examples 1-29 can optionally be configured such that all elements or options recited are available to use or select from.

The above detailed description includes references to the accompanying drawings, which form a part of the detailed description. The drawings show, by way of illustration, specific embodiments that may be practiced. These embodiments are also referred to herein as “examples.” Such examples may include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “tailored,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments may be used, such as by one of ordinary skill in the art upon reviewing the above description. The Abstract is to allow the reader to quickly ascertain the nature of the technical disclosure and is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the embodiments should be determined with reference to the appended claims, along with the full scope of equivalents to which such claims are entitled.

1. An implant assembly comprising:
a primary structure securable to a bone of a patient, the primary structure including:
an adapted portion manufactured to match an anatomy of the bone of the patient and engageable with the bone of the patient; and
a mating portion extending away from the adapted portion; and
an attachment securable to the primary structure, the attachment including:
a coupling portion engageable with the mating portion of the primary structure;
an articular portion opposite the coupling portion, the articular portion defining an implant articular surface, the implant articular surface engageable with a second bone of the patient; and
a tailored portion extending between the coupling portion and the articular portion, the tailored portion manufactured to match the anatomy of the bone of the patient and engageable with the bone of the patient.

2. The implant assembly of claim 1, wherein the implant articular surface is congruent to and matches a bone articular surface of the bone of the patient to together form an improved articular surface engageable with the second bone of the patient.

3. The implant assembly of claim 1, wherein the attachment is made of a vitamin E polyethylene.

4. The implant assembly of claim 1, wherein the attachment includes a post comprising:
a base extending from the coupling portion; and
a distal section extending from the base, wherein the distal section includes a cross-sectional area greater than a cross-sectional area of the base.

5. The implant assembly of claim 4, wherein the primary structure includes an attachment channel engageable with the post of the attachment to secure the attachment to the primary structure.

6. The implant assembly of claim 5, wherein the primary structure further comprises a guide bore, the guide bore configured to receive a fastener to attach the implant assembly to the bone of the patient.

7. The implant assembly of claim 6, wherein the guide bore extends through the primary structure such as to direct the fastener into a bone vault of the bone of the patient.

8. The implant assembly of claim 1, wherein the primary structure comprises a porous region, the porous region configured to attach soft tissue of the patient to the implant assembly.

9. The implant assembly of claim 1, wherein the primary structure comprises a suture canals, the suture canal configured to receive sutures to attach soft tissue of the patient to the implant assembly.

10. The implant assembly of claim 1, wherein the bone is a scapula and wherein the second bone is a humerus.

11. The implant assembly of claim 1, wherein the primary structure is made of OsseoTi®.

12. An implant assembly comprising:
a primary structure securable to a bone of a patient, the primary structure including:
an adapted portion manufactured to match an anatomy of the bone of the patient and engageable with the bone of the patient; and
an attachment securable to the primary structure, the attachment including:
an articular portion defining an implant articular surface, the implant articular surface engageable with a second bone of the patient; and
a tailored portion extending from the articular portion, the tailored portion manufactured to match the anatomy of the bone of the patient and engageable with the bone of the patient.

13. The implant assembly of claim 12, wherein the implant articular surface is congruent to and matches a bone articular surface of the bone of the patient to together form an improved articular surface engageable with the second bone of the patient.

14. The implant assembly of claim 12, wherein the attachment includes a post comprising:
a base extending from a periphery of the attachment; and
a distal section extending from the base, wherein the distal section includes a cross-sectional area greater than a cross-sectional area of the base.

15. The implant assembly of claim 14, wherein the primary structure includes an attachment channel engageable with the post of the attachment to secure the attachment to the primary structure.

16. The implant assembly of claim 15, wherein the primary structure further comprises a guide bore, the guide bore configured to receive a fastener to attach the implant assembly to the bone of the patient.

17. The implant assembly of claim 16, wherein the guide bore extends through the primary structure such as to direct the fastener into a bone vault of the bone of the patient.

18. The implant assembly of claim 12, wherein the primary structure comprises a porous region, the porous region configured to attach soft tissue of the patient to the implant assembly.

19. The implant assembly of claim 12, wherein the primary structure comprises suture canal, the suture canal configured to receive sutures to attach soft tissue of the patient to the implant assembly.

20. A method of making an implant assembly comprising:
determining an anatomical shape of a bone of a patient via a CT scan of the bone of the patient;
forming, with an additive manufacturing machine, an adapted portion of a primary structure such that the adapted portion matches the anatomical shape of the bone of the patient;
determining a shape of an articular portion of the bone of the patient via the CT scan of the bone of the patient; and
machining an attachment of the implant assembly such that a tailored portion of the attachment matches the anatomical shape of the bone of the patient and an articular portion of the attachment matches an articular surface of the bone.

21. The method of claim 20, further comprising:
packaging the primary structure and the attachment of the implant assembly within packaging; and
sterilization the implant assembly and the packaging.

22. A method of inserting an implant assembly into a patient comprising:
retrieving the implant assembly;
placing the implant assembly into a joint of the patient;
positioning the implant assembly against a bone of the patient;
drilling a first hole in the bone of the patient;
drilling a second hole in the bone of the patient;
installing a first fastener through the implant assembly and into the second hole drilled in the bone of the patient; and
installing a second fastener through the implant assembly and the first hole drilled in the bone of the patient.

23. The method of claim 22, further comprising:
opening a package containing the implant assembly; and
coupling a primary structure to an attachment of the implant assembly by inserting a post of the attachment into an attachment channel of the primary structure.

24. The method of claim 22, further comprising pinning the implant assembly within the first hole to hold the implant assembly steady while drilling the second hole within the bone of the patient.

25. The method of claim 22, further comprising attaching soft tissue to the implant assembly by inserting soft tissue through suture canal in the implant assembly.

26. The method of claim 22, further comprising attaching soft tissue to the implant assembly by inserting soft tissue through a porous region of the implant assembly.
